(19) United States
(12) Patent Application Publication
(10) Pub. No.: US 2025/0166801 A1
(43) Pub. Date: May 22, 2025

(54) COMPUTER IMPLEMENTED METHOD FOR PROVIDING A CASE MIX SCHEDULE

(71) Applicant: DEO N.V., Beringen (BE)

(72) Inventors: Jeroen Willem Rita DILLE, Beringen (BE); Niels FESTJENS, Beringen (BE); Clara BALUST MARTINEZ, Beringen (BE)

(73) Assignee: DEO N.V., Beringen (BE)

(21) Appl. No.: 18/729,655

(22) PCT Filed: Jan. 25, 2023
(86) PCT No.: PCT/EP2023/051794

§ 371 Date: Jul. 17, 2024

(30) Foreign Application Priority Data
Jan. 25, 2022 (NL) 2030693

(51) Int. Cl. G16H 40/20; G16H 10/60; G16H 20/40

(52) U.S. Cl. CPC G16H 40/20; G16H 10/60; G16H 20/40

(57) ABSTRACT

A computer implemented method for providing a case mix schedule for patients in at least one operating room includes providing an operating room model including historical patient data comprising at least one patient parameter related to the patient; medical procedure data comprising at least a procedure identifier indicative for the type of procedure and a procedure resource parameter being indicative of the resource requirement of a medical procedure, where the procedure resource parameter is associated with at least one patient parameter; historical operating room sensor data containing at least one sensor parameter measured during a medical procedure associated with at least one patient parameter and/or procedure identifier, providing patient data for the patients; providing operating room sensor data comprising at least one sensor parameter; and providing the case mix schedule for the patients on the basis of the operating room model, the patient data and the operating room sensor data.

The field of the invention relates to a computer implemented method for providing a case mix scheduling for a plurality of patients in at least one operating room. The invention further relates to a computer program product for executing said method and a system for providing said case mix schedule.

Patient scheduling has a direct impact on the organization of the operating room (OR) and the hospital in general. Case scheduling is directly linked to the planning of human, material and infrastructure resources. It also immediately impacts short term impact on OR throughput.

Nowadays, a case mix schedule is typically decided on by experience, by working together with a surgeon for multiple years. Typically, it is an evolving ‘document’, typically in the scheduling nurses head, where historic reasons play a role and maybe even politics. A surgeon will usually push to have as many days as possible (as long as they have enough patients in their backlog; more on that later). Some surgeons might also prefer to have surgery days in the beginning of the week rather than at the end to have patients out of the hospital before the weekend. Sometimes it is best to have two days in between surgery days to make sure that there are enough beds.

This whole puzzle needs to fall together considering all concerned surgeons, for instance making use of one OR or a group of operating rooms, when they have consultation days, their holidays or conference days and making sure that all surgeons get their promised OR time. However, this data evolves over time due to staff rotation, procedural changes, technology changes. In that respect, the resulting case mix schedule may not always be most efficient, for instance in terms of optimal resource usage, while also the process of providing such a scheduling is cumbersome.

The object of embodiments of the present invention is to provide an improved method for providing a case mix schedule.

According to a first aspect of the present invention there is provided a computer implemented method according to claim. More specifically, there is provided a computer implemented method for providing a case mix schedule for a plurality of patients in at least one operating room, wherein the method comprises the steps of:
providing an operating room model, wherein the operating room model comprises:
   historical patient data comprising at least one patient parameter related to the patient;
   medical procedure data comprising at least a procedure identifier indicative for the type of medical procedure and a procedure resource parameter being indicative of the resource requirement of a medical procedure, such as duration and/or other resource requirements of the medical procedure, wherein said procedure resource parameter is associated with at least one patient parameter;
   historical operating room sensor data containing at least one sensor parameter measured during a medical procedure associated with at least one patient parameter and/or procedure identifier;
providing patient data for the plurality of patients, wherein the patient data at least comprises patient parameters for the respective patients;
providing operating room sensor data comprising at least one sensor parameter;
providing the case mix schedule for the plurality of patients on the basis of the operating room model, the patient data and the operating room sensor data.

Accordingly, instead of relying (solely) on the expertise and experience of a planner, a case mix schedule is provided based on a digital operating room model, preferably containing historical patient data, medical procedure data and historical operating room data, which is fed patient data, preferably including at least a patient parameter and a procedure identifier, and operating room sensor data. By using sensor data, a planning can be provided efficiently, with relatively low computational power, as compared to not using sensor data.

Generally, it is preferred if the operating room model contains historical data, for instance based on prior data, wherein the case mix schedule is based on actual or live data, corresponding to the historical data. Preferably, the actual data and the historical data, in particular any parameters therein, correspond to such an extent that comparison, classification and/or correlating the two datasets is possible in order to provide a case mix schedule. As will be explained in greater detail below, the method further comprises the step of including, in said operating room model, the actual data for updating the operating room model.

The case mix schedule, or pre-operative planning, may comprise, for at least one operating room, at least one of a time schedule of the respective medical procedures and preferably required resources, such as personnel, instruments and tools, for at least one day. The case mix schedule may also include a planning for a plurality of operating rooms, wherein preferably also the operating room model contains data for said plurality of operating rooms. The case mix schedule may further include a planning for a plurality of days.

The operating room model preferably comprises historical patient data comprising at least one patient parameter related to the patient. The patient parameter may for instance include a parameter such as age, gender, weight and/or medical history. Preferably, the patient data comprises a plurality of patient parameters and preferably these parameters are grouped or otherwise associated to a single patient. Preferably, the historical patient data thus comprises, for a plurality of patients a plurality of patient parameters. The patient data may for instance comprise a patient identifier, which is preferably not directly linkable to an actual patient, associated therewith a plurality of patient parameters. The patient identifier preferably comprises a randomized identifier.

The operating room model further preferably comprises medical procedure data comprising at least a procedure identifier indicative for the type of medical procedure and a procedure resource parameter being indicative of the resource requirement of a medical procedure, such as duration and/or other resource requirements of the medical procedure. Associated with a type of procedure, as indicated by the procedure identifier, is thus at least one procedure resource parameter. The medical procedure data may also include, next to a duration of the procedure, an active time parameter representative of the duration a resource is required for said medical procedure and a turnaround parameter, representative of the required turnaround time between procedures wherein said resource is not required, as will be explained in greater detail below.

The medical procedure data may comprise historical procedure data obtained from previous procedures. The medical procedure data preferably comprises data for a plurality of medical procedures, each having associated therewith a procedure resource parameter. It may be the case that for one type of procedure, which may thus have the procedure identifier, the medical procedure data comprises a plurality of different procedure resource parameters. It is then preferred if the procedure resource parameter is further associated with at least one patient parameter, or for instance a patient identifier as mentioned above.

In that case, it is possible to take into account, when providing the case mix schedule for a patient parameter of the patient data, a procedure resource parameter associated with said patient parameter. It may for instance be the case that a particular procedure, for instance defined or identifiable by a procedure identifier as mentioned above, requires different resources for different patient parameters. The case mix schedule for a patient, next to other patients in the patient data, having a patient parameter is then preferably based on a procedure resource parameter associated with, or at least similar to, said patient parameter.

By having knowledge on the historical impact of case parameters on relevant operating room parameters, it then is possible to predict those values for different patients and procedures. For instance, data could show the duration of a HIP revision surgery on an 85 year old obese patient is typically longer and has a much higher standard deviation of duration than a primary hip replacement on a 62 year old non-obese patient.

Additionally, or alternatively, the medical procedure data may comprise a plurality of predetermined procedure identifiers, each indicative for the type of medical procedure, and a predetermined procedure resource parameter being indicative of the resource requirement of a medical procedure associated therewith. The medical procedure data may for instance be based on best practice data and/or otherwise predefined data.

The medical data may also contain procedural data, preferably including required resources as for the medical procedure data, for procedures relating to the medical procedures. A medical procedure in the form of surgery may for instance require pre-clinical procedures or tests. Also, these related procedures are preferably associated with a medical procedure in the medical procedure data such that also these procedures can be efficiently planned.

The medical procedure data may further comprise related procedure data, for instance relating to pre-clinical and/or pre-diagnostic procedures. At least one related procedure, preferably associated with a related procedure identifier, may thus be associated with a medical procedure in the medical procedure data. Also, the related procedure data preferably comprises for a plurality of related procedures, preferably identifiable by the related procedure identifier, at least one related procedure resource parameter, indicative of the required resources of the related procedure, such as duration, personnel and/or required instruments or tooling. The related procedure resource parameter, for a related procedure, may again be associated with a patient parameter. Different patients, as reflected by the patient parameter, may require different resources.

Preferably, the step of providing the case mix schedule then also takes into account any associated related procedures. The schedule may then include a planning for the related procedures and the available resources therefore.

The operating room model further preferably comprises historical operating room sensor data containing at least one sensor parameter measured or obtained on the basis of data obtained during a medical procedure. Sensor data may include data measured by measurement means during a medical procedure, such as audio and/or video measurements, medical device readout (such as hearth rates, blood pressures etc.), but preferably also includes usage statistics of tools. The sensor data may also include data obtained from for instance wearables and may generally comprise presence data of personnel or usage data of the instruments. These usage statistics may be determined from sensor data obtained during a medical procedure. The sensor data may also include received procedural (log) data such as starting time and/or end time of a procedure, or even of sub steps of a procedure, as will be explained below. The historical operating room sensor data may assist, on the basis of operating room sensor data, in more accurately defining the case mix schedule and/or in updating the planning, while also requiring less resources to provide an accurate planning.

The at least one sensor parameter is preferably associated with at least one patient parameter and/or procedure identifier, preferably as defined in the historical patient data or the medical procedure data. On the basis of a provided sensor parameter in the operating room sensor data, the case mix schedule may then be based on procedure resource parameters associated with said patient parameter and/or procedure identifier as associated with the sensor parameter in the historical operating room sensor data. New insights from the operating room, in the form of operating room sensor data, may then contribute to defining or updating the case mix schedule.

The step of providing the case mix schedule preferably comprises outputting, for instance using a suitable output unit, the case mix schedule. The case mix schedule is preferably made available using digital means. Such an output unit may for instance be embodiment in a server or a plurality thereof.

Preferably, providing the case mix schedule comprises providing the planning to digital hospital management system. The hospital management system may comprise data concerning the scheduled patients, which may be updated based on the provided case mix schedule. Preferably, the case mix schedule is further at least partly based on information or data from the hospital management system. The case mix schedule may for instance be based on occupation rate of the hospital, staff availability, related procedure availability (see above), instrument availability and/or other logistic parameters.

According to further preferred embodiment, the operating room model comprises hospital specific data, wherein said hospital specific data comprises hospital parameters such as available beds, available operating rooms or other medical facilities. Preferably, the case mix schedule is also based on the hospital specific data. The method may comprise the step of obtaining the hospital specific data, for instance from a hospital management system or general ERP system as mentioned above.

Preferably, the medical procedure data further comprises a stay parameter indicative of the length of stay associated with a medical procedure. Preferably, said stay parameter is also associated with at least one patient parameter, preferably in combination with a procedure identifier. On the basis of the type of patient, and preferably type of procedure, a length of stay can be predicated from said data. Providing the planning may thus include providing a planning of the predicted lengths of stay of the respective patients. For instance, based on the available beds in the hospital, a planning can be provided, taking into account the predicated required beds in the following days.

Also here, the medical procedure data in the operating room model is preferably updated on the basis of received data. A measured length of stay, for instance following from the sensors data, of a patient can be included in the medical procedure data, preferably associated with other parameters and/or identifiers.

It should be noted that although reference is made to an operating room model and operating room sensor data, these features may encompass more than data or sensor data obtained from the operating room only. Generally, the operating room model may be considered as a medical simulation model, including and making use of medical sensor data in general, i.e. not only originating from the operating room.

Preferably, the step of providing operating room sensor data comprises measuring operating room sensor data is said operating room. The sensor data may for instance include audio and/or video data. The method then preferably comprises determining, from said sensor data said at least one sensor parameter.

It may be possible that live operating room sensor data is used, for instance fed to the processing unit as will be explained below, to provide or update the case mix schedule. It may however also be possible that the planning is provided or updated intermittently on the basis of sensor data.

The method may further include the step of comparing the sensor parameters of the obtained operating room sensor data and historical operating room sensor data. The data may be obtained in the current hospital, or may also include data obtained from other hospitals. A sensor parameter as received may thus be compared to any sensor parameter in the historical data. If any significant differences are detected, an updated case mix schedule may be provided. The method then preferably comprises the step of, in dependence on a measured difference, updating the case mix schedule. The method may also include the step of only providing the case mix schedule if a predetermined difference is determined between the sensors parameters.

The method may also include, following a measured difference as mentioned above, updating the operating room model on the basis of the operating room sensor data. Thus, following a discrepancy between expected sensor parameters and actual sensor parameters, the model may be updated accordingly. The step of providing a planning may then be dispensed with and a method for providing an operating room model may be provided according to a further aspect, as will be explained below.

Updating or providing the pre-operative plan may also take place without including the receipt of sensor data. The operating room model may then also lack sensor data.

It may also be the case that an optimal case mix schedule is provided for a plurality of patients having generic patient parameters and requiring generic medical procedures of different types. A planning may then be provided on the basis of this data and the operating room model. A more generic case mix schedule may thus be provided, on the basis of which the medical procedures of the actual patients can be planning, wherein the patient parameters and required procedures of these patient correspond at least to a large extent to the generic patient parameters. This generic planning may be intermittently updated, perhaps on the basis of operating room sensor data.

According to a preferred embodiment, the step of providing the case mix schedule comprises updating, on the basis of an updated operating room model and/or operating room sensor data, the case mix schedule. A previously provided planning is thus updated. The previously provided planning may, as will be explained below, also be included in the operating room model, in particular if the planning is corrected for actual resources.

Preferably, the case mix schedule is updated on the basis of measured operating room sensor data, the medical procedure data, the historical patient data and/or the historical operating room sensor data. A measured change in the operating room, for instance a detection that a procedure is terminated prior to the planning, may for instance result in the update of the planning.

In order to improve the quality of care, next to an improved planning, it is preferred to also include parameters defining the quality and/or outcome of a medical procedure. These parameters may for instance be obtained from the patient, the surgeon, OR personnel and/or any medical practitioner involved in the post operative process. The data may be obtained during a follow-up of the medical procedure. The data may thus be updated on the basis of data obtained after several days, weeks or even months after said medical procedure. Thus, preferably, the medical procedure data comprises, for a plurality of medical procedures, an historical parameter being indicative of the outcome of a medical procedure, such as success and/or duration of the medical procedure. A medical procedure, next to a procedure identifier to identify the type of procedure as mentioned above, may thereto have a unique procedure identifier. Also associated herewith may be the historical parameter. As such, it is known which historical procedure has which outcome, as reflected by the historical parameter.

Preferably, said historical parameter is associated with at least one patient parameter and/or procedure identifier. For a given patient group, based on the patient parameter and the procedure identifier, a prediction can thus be made of the outcome of the procedure. This is preferably taken into account when providing the planning.

Preferably, at least one sensor parameter measured during a medical procedure is associated with at least one patient parameter and an historical parameter. This allows predicting the outcome of the procedure, for instance in terms of duration and/or success, on the basis of measured operating room sensor data.

As mentioned above, the medical procedure data may comprise both historical data, reflecting past procedures and associated resources and/or outcomes, and generic medical procedure data, for instance based on best practice methods. Based on the type of procedure and the patient group, an optimal case mix schedule can thus be provided.

It is however preferred if the operating room model further comprises process map data indicative of steps, including possible alternative steps, for a type of medical procedure. In the model, a medical procedure is thus divided in a plurality of steps, including any alternative steps. In certain medical procedures, as defined by the procedure identifier, different approaches can be used to obtain a goal. These different approaches are then included in the model as alternative steps. As such, the process map may comprise a tree-like or flow chart structure with a plurality of alternative steps.

To identify these steps, the process map data preferably comprises, associated with at least a procedure identifier, a plurality of steps and hierarchical data representative of the hierarchical order of said steps. This data preferably defines the process map data as mentioned above and may form a tree-like structure with the different steps.

Preferably, at least one step resource parameter is associated with at least one step indicative of a resource requirement, such as duration and/or required tooling, for said step. The resource parameter associated with a step may for instance reflect the time needed for a certain step.

The case mix schedule is then preferably also based on the process map data. By taking into account the different steps in a certain procedure, a more accurate planning can be made. It is then preferred that the steps, in particular any preferred steps of alternative steps, are also included in the planning.

Preferably, the process map data further comprises probability parameters associated with at least two alternative steps. The probability parameters define the probability that a certain step is required in a certain procedure. Taking this probability, for instance together with the resource requirement for a certain step, into account allows a more accurate and more efficient planning.

Preferably, said probability parameter is associated with a patient parameter. This allows planning on the basis of the received patient data including said patient parameter, while taking into account the probability of said patient if a certain alternative step is likely to be required.

Preferably, said probability parameter is associated with at least one sensor parameter. A sensor parameter, for instance a certain type of sound or the usage of a device in the operating room or an event detected by a deep-learning algorithm on an image stream, may be indicative of the chance of occurrence of an alternative step. Upon receiving any operating room data including said sensor parameter, an updated planning may be provided.

According to a preferred embodiment, the operating room model comprises a predictive model. On the basis of the patient data and in particular the medical procedure data containing historical data, a prediction can be made of the optimal planning for an operating room, or a plurality thereof. On the basis of the received data, a predication may be made regarding which steps for a medical procedure are likely needed, based for instance of the probabilities as mentioned above.

According to a preferred embodiment, the step of providing the pre-operative plan comprises providing an optimization parameter of the plurality of parameters in said operating room model, wherein the step of providing the case mix schedule comprises minimizing said optimization parameter in said operating room model on the basis of the received data. The step of providing the case mix schedule may for instance comprise solving a minimization or optimization problem. By properly defining the objective function, for instance a reduced number of resources, the problem, on the basis of the operating room model can be solved to minimize the objective function.

The step of providing the case mix schedule on the basis of the operating room model may comprise techniques of operations research such as integer programming, dynamic integer programming, and combinatorial optimization.

The operating room model may also contain, for instance associated with the medical procedures, cost information regarding the medical procedures and any resources. This allows taking into account the costs aspect in planning. For instance, in a minimization or optimization problem as mentioned above, the objective function may include a cost aspect or may generally be arranged to reduce costs when providing the planning.

Preferably, the method further comprises the steps of updating the operating room model on the basis of the received operating room sensor data comprising the at least one sensor parameter. The received operating room sensor data including the sensor parameter is then preferably added to the historical operating room sensor data of the operating room model. Also, a patient parameter, as received in the patient data, and/or a procedure identifier, as obtained for instance for the provided case mix schedule, is then preferably associated with said sensor parameter upon including this data in the historical operating room sensor data.

Preferably the step of providing the operating room model comprises further comprising the step of providing an operating log indicative of the plurality medical procedures on the plurality of patients in said operating room and determining, on the basis of said operating log, said procedure resource parameters. Said operating log may for instance be based on the case mix schedule and may reflect the actual schedule of the day or plurality of days. The log may also be determined, at least partly, on the basis of operating room sensor data as mentioned above. Any start and end times, for instance of certain procedural steps, may for instance be determined from the operating room sensor data.

According to a preferred embodiment, the medical procedure data comprises, for a plurality of medical procedures preferably associated with a procedure identifier, an active time parameter representative of the duration a resource is required for said medical procedure and a turnaround parameter, representative of the required turnaround time between procedures wherein said resource is not required, wherein the step of providing the case mix schedule comprises providing, in said planning, predicted active time parameters and turnaround parameters for the at least one operating room. This allows providing the planning is an efficient way. the active time parameter may for instance be representative of the required presence of a surgeon or other resource, while the turnaround parameter is indicative for the time between active surgery, for instance including preparation and finishing up the procedure, for which the presence of a surgeon is not required.

Preferably, the method further comprises the step of providing, on the basis of the active time parameters and the turnaround parameters for a planning, an active time ratio indicative of the ratio of the active time parameter and the turnaround parameter, wherein the step of providing the planning comprises optimizing for the active time ratio. As an example, when the ration is lower than one, the resource is not used optimally. Dependent on the resource, the planning may thus be updated to increase the active time ratio.

Preferably, a medical procedure may have associated therewith a plurality of active time parameters and turnaround parameters for a plurality of resources, for instance identifiable by a resource identifier. The resources may be associated with a weight factor, wherein the planning is based on the respective active time parameters, preferably the ratio as mentioned above, and the associated weight. Different resources may then be taken into account when optimizing the planning. Idle time of the surgeon may for instance be less preferred than idle time of an operating assistant. Both may however be taken into account.

Preferably, the active time parameters and turnaround parameter are associated with at least two steps of the process data as mentioned above. This allows providing a planning in an efficient manner.

According to a further preferred embodiment, the method comprises determining from the operating room sensor data the active time parameter and/or the turnaround time parameter and including said parameters in the operating room model. Sensor data from the operating room may for instance include presence or use data, indicative for the presence or use of a resource. The data may include, for instance as a parameter, the determined surgical time (skin to skin) or equipment usage. Preferably, the data is included in the operating room model, associated with a procedure identifier and most preferably a patient parameter.

According to a further aspect, there is provided a method for providing an operating room model, in particular for use the method as described above, wherein the method comprises the steps of:
providing historical patient data comprising at least one patient parameter related to the patient;
providing medical procedure data comprising at least a procedure identifier indicative for the type of medical procedure and a procedure resource parameter being indicative of the resource requirement of a medical procedure, such as duration and/or other resource requirements of the medical procedure, wherein said procedure resource parameter is associated with at least one patient parameter;
providing historical operating room sensor data containing at least one sensor parameter measured during a medical procedure associated with at least one patient parameter and/or procedure identifier, and
including the historical patient data, the medical procedure data and the historical operating room sensor data in an operating room model.

As mentioned, the method may include the step of updating the operating room model, for instance based on newly received data and/or when received sensor parameters differ from sensor parameters in the historical operating room sensor data.

According to a further aspect, there is provided a computer program product comprising a computer-executable program of instructions for performing, when executed on a computer, the steps of the method as defined above.

According to a further aspect, there is provided a system for providing a case mix schedule for a plurality of patients in at least one operating room, preferably according to the method as described above, wherein the system comprises:
a model storage unit arranged for storing an operating room model, wherein the operating room model comprises:
   historical patient data comprises at least one patient parameter related to the patient;
   medical procedure data comprising at least a procedure identifier indicative for the type of medical procedure and a procedure resource parameter being indicative of the resource requirement of a medical procedure, such as duration and/or other resource requirements of the medical procedure, wherein said procedure resource parameter is associated with at least one patient parameter;
   historical operating room sensor data containing at least one sensor parameter measured during a medical procedure associated with at least one patient parameter and/or procedure identifier;
a patient data input unit arranged to receive patient data for the plurality of patients, wherein the patient data at least comprises patient parameters for the respective patients;
a sensor data input unit arranged to receive operating room sensor data comprising at least one sensor parameter;
a processing unit arranged to provide the case mix schedule for the plurality of patients on the basis of the operating room model, the patient data and the operating room sensor data;
an output unit for outputting the case mix schedule.

The model storage unit, the data inputs units, the processing unit and the output unit may be formed by respective computers, other processing means such as servers, and/or at least some of the units may be part of a single server or computer. As mentioned, the system is arranged to perform the method as described above.

The accompanying drawings are used to illustrate presently preferred non-limiting exemplary embodiments of devices of the present invention. The above and other advantages of the features and objects of the present invention will become more apparent and the present invention will be better understood from the following detailed description when read in conjunction with the accompanying drawings, in which:

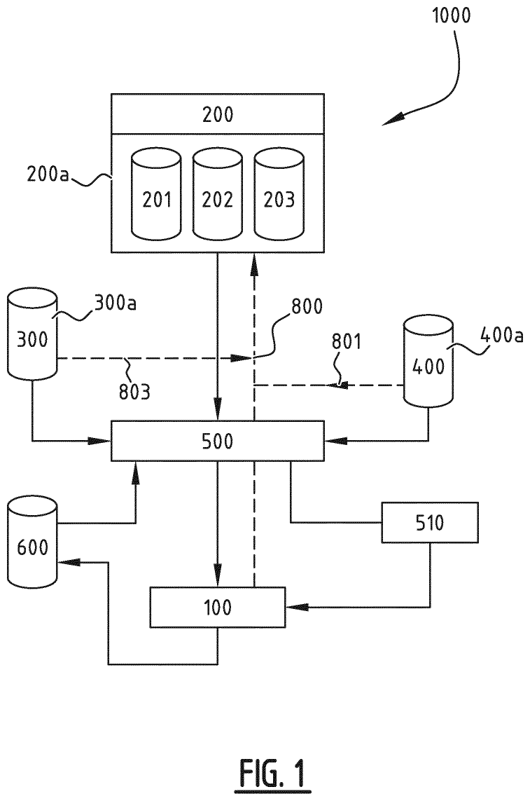

FIG. 1 schematically shows an overview of the method;

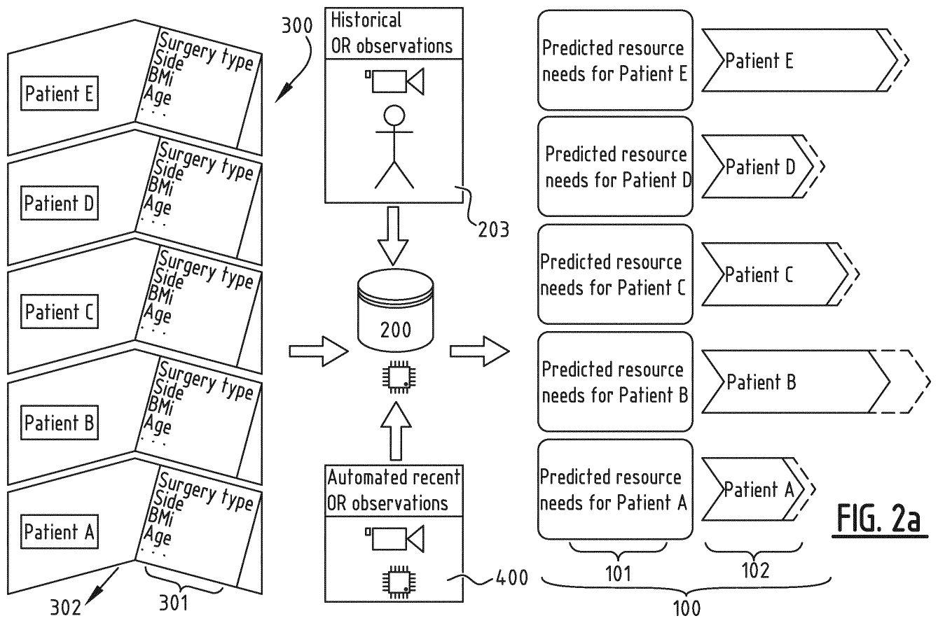

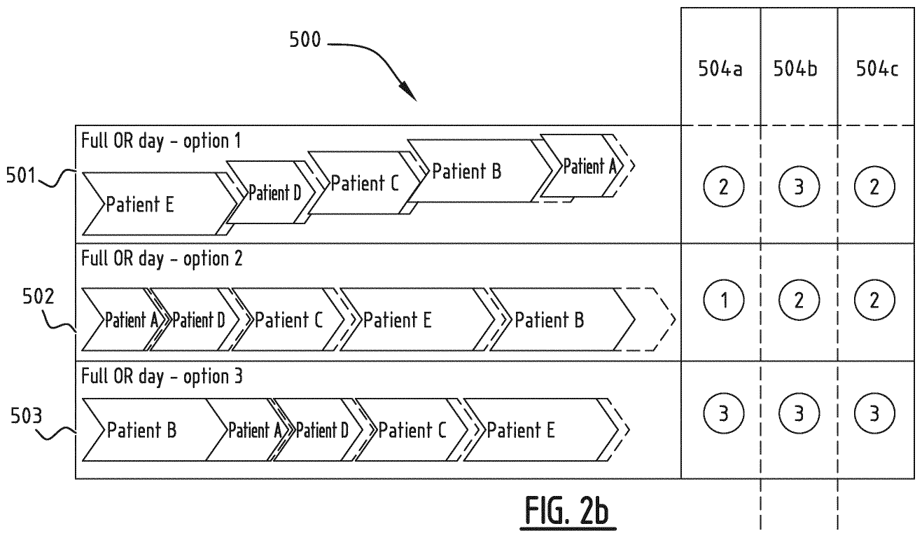

FIGS. 2a and 2b schematically show an example of a planning for four patients;

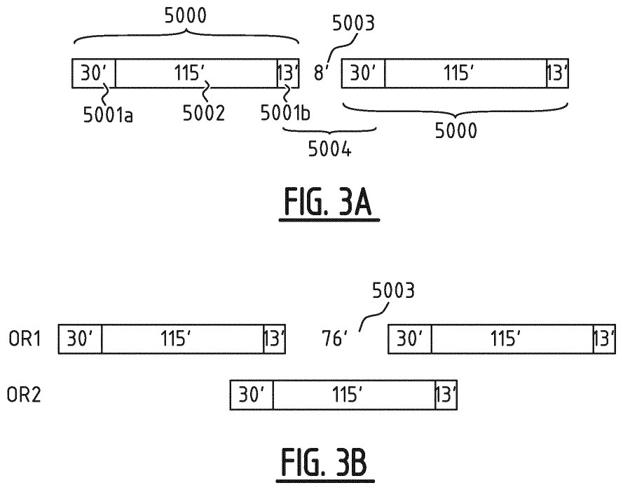

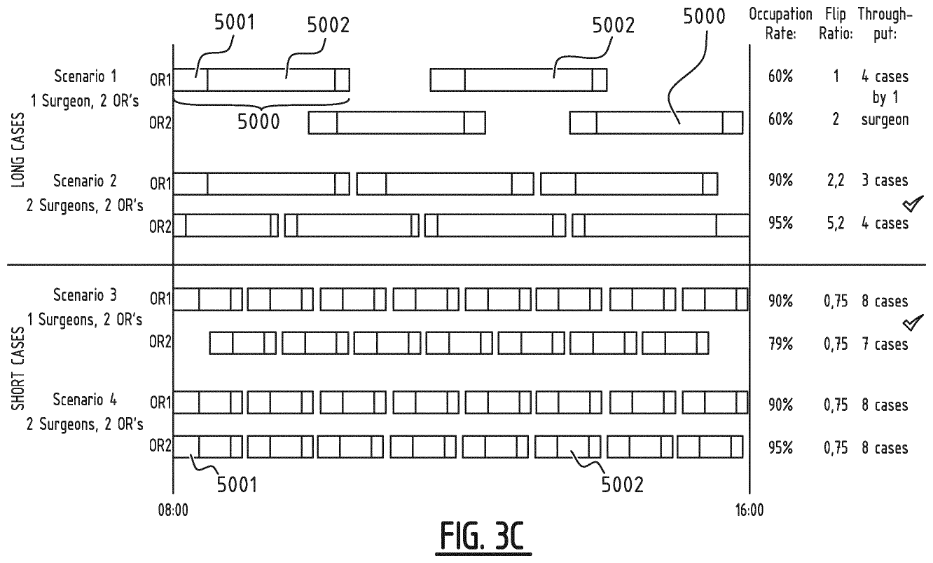

FIGS. 3a-c show additional examples of a different planning;

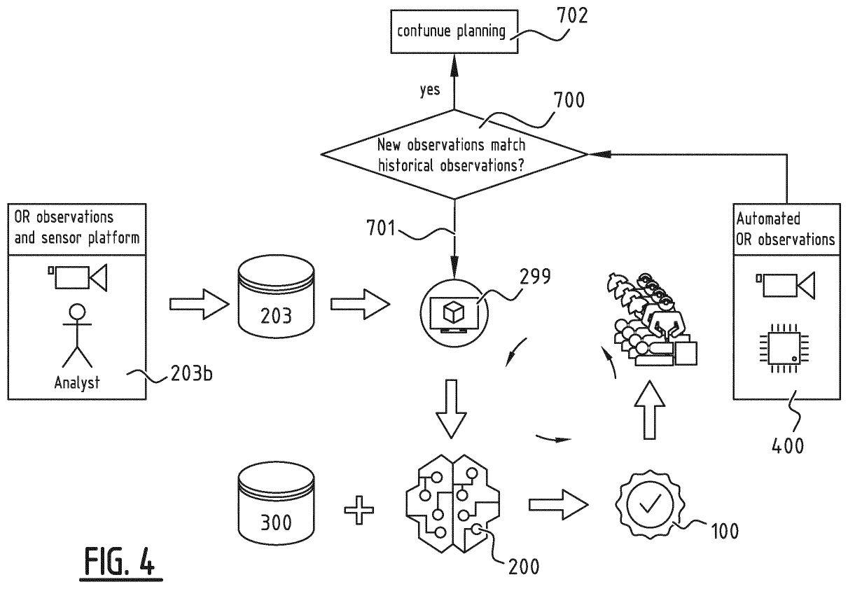

FIG. 4 schematically shows an example of the updating process of the method;

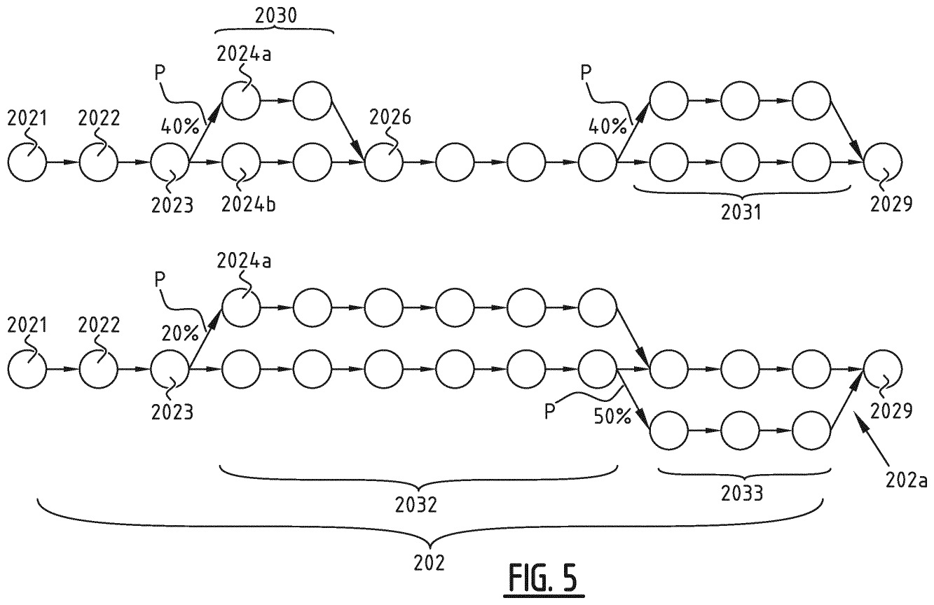

FIG. 5 shows two examples of process map data of medical procedures.

In FIG. 1, a method 1000 for providing a case mix schedule 100 for a plurality of patients in at least one operating room. The method comprises the step 200a of providing an operating room model 200, wherein the operating room model comprises historical patient data 201 comprising at least one patient parameter related to the patient, medical procedure data 202 comprising at least a procedure identifier indicative for the type of medical procedure and a procedure resource parameter being indicative of the resource requirement of a medical procedure, such as duration and/or other resource requirements of the medical procedure and historical operating room sensor data 203 containing at least one sensor parameter measured during a medical procedure associated with at least one patient parameter and/or procedure identifier.

The method further comprises the step 300a providing patient data 300 for the plurality of patients which are to receive medical treatment The patient data 300 comprises data, parameters 301 (see also FIG. 3a), about the patients, such as surgery type, side, BMI and age, next to a unique patient identifier 302. The same parameters are included in the patient model 200. The patient data 300 may for instance correspond to the electronic patient records (EPD) of the patients listed for planning.

The method further comprises the step 400a of providing operating room sensor data 400 comprising at least one sensor parameter. The operating sensor data 400 comprises observations (sensor parameters) obtained in the operating room. To determine the sensor parameters, a combination of different sensors and technologies can be used. Possible parameters are computer vision to map staff presence, OR activities, ergonomics and instrument usage. This can be used to calculate several parameters such as case duration, ergonomics and instrument requirements per case, including the variability on those parameters. This can optionally be combined with other sensors such as Bluetooth or RFID sensors to track equipment, track personnel and patients, heart rate sensors to measure stress levels. The parameters in the operating room sensor data 400 preferably correspond to the data included in the historical operating room sensor data 203.

The method then comprises the step 500 of providing the case mix schedule 100 for the plurality of patients on the basis of the operating room model 200, the patient data 300 and the operating room sensor data 400.

By providing an operating room model 200, which may correspond to a digital twin of an operating room, an optimal case mix schedule 100 may be provided. In the step 500 of providing the planning 100, account can be taken of different aspects in terms of planning optimization.

In the medical procedure data 201, required resources are preferably stored. These resources may include OR usage, such as procedure duration, staff requirements and instrument requirements. The medical procedure data 201 further preferably comprises a historical parameter being indicative of the outcome of a medical procedure, such as success and/or duration of the medical procedure. On the basis of this information, a planning 100 can be provided, which includes a planning 101 in terms of resources (such as instruments) and expected duration of the procedures, indicated with 102 in FIG. 2a.

The step 500 of providing a planning may comprise providing a plurality of plannings 501-503 as shown schematically in FIG. 2b. In this figure, three different options for a planning are shown, wherein each planning is based on the required resources 101 and 102 as shown in FIG. 2a.

The method may thus comprise an optional step 510 (see FIG. 1) of optimizing the planning 100. The optimization can be based on several parameters, schematically indicated with 504a-c in FIG. 2b, each having a score (in this example ranging from 1 to 3) for each parameter. In this example, the first parameter includes staff availability including ergonomics, the second parameter 504b includes instrument availability and the third parameter 504c includes patient comfort. Also based on the importance of the three parameters 504a-c, for which weighing factors may be used, a preferred planning can be determined. In this example, planning 503 is preferred and will as such be outputted as the case mix schedule 100.

Next to the above-mentioned data files, additional data 600 (see FIG. 1) can be used such as patient record systems for receiving the right details on the pathology of the patient, ordering systems, Human Resources systems, Enterprise Resource Planning systems and Central Sterilization Unit management systems. At the same time, the case mix schedule 100 can be pushed to OR management systems and/or patient case scheduling systems, next to the above-mentioned systems.

With reference to FIGS. 3a-c, providing a planning based on active time parameters and turnaround parameters is explained. The active time ration, or the Flip Ratio is a parameter designed to help assess whether a planning is optimal. The parameter can for instance be used optimization parameter or objective function in an optimization problem. The ratio is indicative for the efficiency in terms of planning for a particular procedure, surgeon, and the operating room's operational setup. It may help in determining whether it is possible for the surgeon to perform procedures in ORs in parallel or not.

While the Flip Ratio described here is in relation to the surgeon, the concept also applies to other members of the OR staff or generally other resources. The Flip ratio is calculated by dividing the total time that the surgeon is required to be in the OR (active time parameter) by the total time that the surgeon is not required to be in the OR (turnaround time parameter). The time taken by a surgeon and his/her team to perform the procedure is a function of the type of procedure and may be contained in the medical procedure data. It is also a function of the OR's operational efficiency and the respective SOPs (standard operating procedure) in place.

In FIG. 3a, the boxes 5000 represents each patient in the room, and the boxes 5002 represent the surgeon time (in this case skin to skin). Boxes 5001a and 5001b represent the preparation time (patient preparation) and the time required for finishing up the procedure (patient breakdown), for which the presence of the surgeon is not required. The area 5003 gray between the boxes 5000 is the time between procedures. The time indicated with 5004 is thus the time the surgeon is not required. This time will be called the turnaround time, for which the turnaround time parameter is representative.

In FIG. 3A, there is a surgeon operating in one OR only, the Flip ratio becomes:

flip ratio=(surgeon time)/(no surgeon time)=115/(30+13+8)=2.25

no surgeon time=patient preparation+patient breakdown+turnaround

In this case (for this procedure type, with this level of efficiency, with this OR model, etc), the Flip ratio is more than 1. This value indicates that if the surgeon would perform the same procedure in two ORs in parallel (thus Flipping between two ORs), the occupational rate of the OR will suffer. This is illustrated in the example of FIG. 3b, which illustrates a day with cases being performed in parallel OR's. In this case, the ratio=(surgeon time)/(no surgeon time)=115/(30+13+76)=0.96

In this case, the flip ratio is almost 1. When having parallel OR's, the Flip Ratio will be maximum 1. Whenever the Flip Ratio is almost 1, one should then look at the occupation rate of the OR, and assess whether the resulting occupation rate of the OR is acceptable. The acceptable threshold will vary from hospital to hospital. In this example, the two OR's have a much lower occupational rate than in the example of FIG. 3a.

Thus, in this way, based on the Flipping ratio value calculated for the procedure type, the planning may be changed if the predicted or observed occupation rate is not acceptable. In this case, a more efficient OR with a higher Occupational rate can be achieved by having the surgeon not have parallel OR's for this type of procedure.

FIG. 3c shows other different scenarios for different procedures for longer procedures on top and shorter on the bottom. The operating room is a scarce and expensive resource, so one should aim at having a continuous flow of patients with as minimum idle time as possible. The goal should thus be to have the highest Occupation Rate possible (percentage of time that a patient is in the OR).

At the same time, one should also minimize the waiting time of the staff members (mainly for surgeons and anesthesiologists).

Each row is the day overview for an OR, the blocks 5000 represent when a patient is in the OR, and the overlapping blocks 5002 again represent when the surgeon is expected to be in that same OR treating the patient. In FIG. 3, four different scenarios are shown: two scenarios with long cases and two scenarios with short cases:
Scenario 1 depicts 1 surgeon doing long cases in 2 OR's (switching from room to room). This has a daily throughput of 4 cases done by 1 surgeon (last case 5000 done shown with dashed lines) and an occupation rate of 60%.
Scenario 2 depicts 2 surgeons (1 surgeon per room). This setting yields a throughput of 7 cases in total (3 and 4 per surgeon, respectively), with an occupation rate in each OR above 90%.

While in scenario 2, surgeon 1 will do one less case, the overall numbers are much better given that the resources are used better. The surgeons will have some waiting time from case to case, but this is holistically better than having 2 OR's half empty during the whole day, just to have a surgeon be operating non-stop.

Scenario 3 shows 1 surgeon working in 2 rooms and doing very short cases. Because the cases are short, and the proportionally the surgeon is needed for about half the time the patient is in the OR, it makes sense to have 2 rooms per surgeon. This yields a daily throughput of 15 cases with also a high occupation rate, and a surgeon with minimal lag time.

Scenario 4 shows the same short cases, but now done with 2 surgeons (each in their own room). This yields (as in scenario 2) a high occupation rate in both OR's, but a very similar throughput than in scenario 3 (only 1 more case is achieved, while 2 senior surgeons are required), with some surgeon lag time throughout the day.

In the short cases day, given the small difference in throughput, it is recommended to have a surgeon flip between rooms as this gives a high throughput with minimal lag time.

In FIG. 4, an example of an updating scheme of the planning 100 and model 200 is shown. In this example, a planning 100 is provided on the basis of patient data 300, for instance containing data obtained from electronic patient records (EPD) and the operating room model 200. Also here, the model 200 is based on historical sensor data 203 which is obtained from historical measurement 203b in the operating room. In this process, a step 700 of comparing the sensor parameters of the obtained operating room sensor data 400 and historical operating room sensor data 203 and, in dependence on a measured difference (step 701), updating the model 299 and/or the case mix schedule 100. In the alternative, the planning may continue (step 702).

As mentioned above, the operating room model 200 comprises medical procedure data 202 comprising at least a procedure identifier indicative for the type of medical procedure and a procedure resource parameter being indicative of the resource requirement of a medical procedure, such as duration and/or other resource requirements of the medical procedure. On the basis of this data 202 and for instance the patient data 300 which preferably also contains a procedure identifier (see surgery type as patient parameter 301 in data 300 in FIG. 2a), an estimate can be provided of the expected resources required, for instance in terms of duration and required tooling (see 101 and 102 in FIG. 2a). Also other parameters, such as outcome or patient comfort can be included in the medical procedure data 202. The respective parameters are preferably associated with at least on patient parameter or a patient identifier form the historical patient data 201. The planning, for the instance the step 501 of providing a plurality of plannings as shown in FIG. 2b, can then be based on the respective parameters associated or similar to the parameters of the patient data 300 for which the planning 100 is to be determined in step 500. Providing a planning 100 (step 500) may then comprise basing a planning 100 for a patient having a patient parameter on a resource parameter associated with or similar to the patient parameter in the medical procedure data 202.

A medical procedure can be subdivided in a plurality of steps. With reference to FIG. 5, the medical procedure data 202, or the operating room model 200 in general, therefore preferably comprises process map data 202a indicative of steps 2021-2029, including possible alternative steps 2024a, 2024b, for a type of medical procedure, wherein the process map data 202a comprises, associated with at least a procedure identifier, a plurality of steps, hierarchical data representative of the hierarchical order of said steps and at least one step resource parameter associated with at least one step indicative of a resource requirement, such as duration and/or required tooling, for said step, wherein the case mix schedule in step 500 is also based on the process map data.

During a medical procedure, several alternatives are typically available. For instance, to keep a wound open during surgery, retractors are used to retract the soft tissue. In many cases, assistants will hold these retractors to keep the wound open. This essentially means that there is one person in the room whose only/main purpose is to hold a retractor. This can be reflected in the resource parameter for this first alternative 2024a. On the other hand, nowadays there are also other systems that allow a stable retractor positioning without the need of a human to hold said retractor. This is for example indicated with step 2024b. As this device does not require an additional person in the operating room, this is reflected in the resource parameter associated with step 2024b. In this case, looking only at the cost side, one can justify that the cost of the single use materials is less than the cost of having one additional person in the OR.

With reference to upper process map, the process map data 202a may contain different alternative paths 2030 of steps which may for instance join, see step 2026. A second branch 2031 may occur later in the procedure. In the lower process map associated with a different type of medical procedure, and thus with a different procedure identifier, the branches 2032 and 2033 may only join at the end 2029. The process map data 202a defines the hierarchy of the different steps 2021-2029.

Although some of the alternative steps are optional and may be decided upon by resource management, steps may also result in complications. It is thus preferred if at least some of the alternative steps have associated therewith a probability P. This is for instance indicated for the steps 202a, 2024b which may follow step 2023. In the above process map, there is a probability of 40% that step 2023 will be followed by step 2024a. A same probability applies for instance in the second branch 2031 as indicated with the letter P.

The probabilities may vary for different groups of patients. Alternative steps may thus have associated therewith a plurality of probabilities, preferably each of which is associated with at least one patient parameter. Dependent on a patient parameter, which may be retrieved from the patient data 300 as provided, different probabilities may thus apply. Taking these probabilities into account when providing the case mix schedule 100 thus improves the planning process.

Again with reference to FIG. 1, it is preferred when the operating room model 200 is updated. Although this can be done according to the scheme explained in FIG. 4, it is preferred if patient data 300 and the operating room sensor data 400 as used in the determination step 500 is also fed back to the operating room model 200. This is schematically indicated with the dashed lines 800, 801 and 803.

A person of skill in the art would readily recognize that steps of various above-described methods can be performed by programmed computers. Herein, some embodiments are also intended to cover program storage devices, e.g., digital data storage media, which are machine or computer readable and encode machine-executable or computer-executable programs of instructions, wherein said instructions perform some or all of the steps of said above-described methods. The program storage devices may be, e.g., digital memories, magnetic storage media such as a magnetic disks and magnetic tapes, hard drives, or optically readable digital data storage media. The program storage devices may be resident program storage devices or may be removable program storage devices, such as smart cards. The embodiments are also intended to cover computers programmed to perform said steps of the above-described methods.

The description and drawings merely illustrate the principles of the present invention. It will thus be appreciated that those skilled in the art will be able to devise various arrangements that, although not explicitly described or shown herein, embody the principles of the present invention and are included within its scope. Furthermore, all examples recited herein are principally intended expressly to be only for pedagogical purposes to aid the reader in understanding the principles of the present invention and the concepts contributed by the inventor(s) to furthering the art, and are to be construed as being without limitation to such specifically recited examples and conditions. Moreover, all statements herein reciting principles, aspects, and embodiments of the present invention, as well as specific examples thereof, are intended to encompass equivalents thereof.

The functions of the various elements shown in the figures, including any functional blocks labelled as “processors” or “units”, may be provided through the use of dedicated hardware as well as hardware capable of executing software in association with appropriate software. When provided by a processor, the functions may be provided by a single dedicated processor, by a single shared processor, or by a plurality of individual processors, some of which may be shared. Moreover, explicit use of the term “processor” or “controller” should not be construed to refer exclusively to hardware capable of executing software, and may implicitly include, without limitation, digital signal processor (DSP) hardware, network processor, application specific integrated circuit (ASIC), field programmable gate array (FPGA), read only memory (ROM) for storing software, random access memory (RAM), and non volatile storage. Other hardware, conventional and/or custom, may also be included. Similarly, any switches shown in the figures are conceptual only. Their function may be carried out through the operation of program logic, through dedicated logic, through the interaction of program control and dedicated logic, or even manually, the particular technique being selectable by the implementer as more specifically understood from the context.

It should be appreciated by those skilled in the art that any block diagrams herein represent conceptual views of illustrative circuitry embodying the principles of the present invention. Similarly, it will be appreciated that any flowcharts, flow diagrams, state transition diagrams, pseudo code, and the like represent various processes which may be substantially represented in computer readable medium and so executed by a computer.

It should be noted that the above-mentioned embodiments illustrate rather than limit the present invention and that those skilled in the art will be able to design alternative embodiments without departing from the scope of the appended claims. In the claims, any reference signs placed between parentheses shall not be construed as limiting the claim. The word “comprising” does not exclude the presence of elements or steps not listed in a claim. The word “a” or “an” preceding an element does not exclude the presence of a plurality of such elements. The present invention can be implemented by means of hardware comprising several distinct elements and by means of a suitably programmed computer. In claims enumerating several means, several of these means can be embodied by one and the same item of hardware. The usage of the words “first”, “second”, “third”, etc. does not indicate any ordering or priority. These words are to be interpreted as names used for convenience.

In the present invention, expressions such as “comprise”, “include”, “have”, “may comprise”, “may include”, or “may have” indicate existence of corresponding features but do not exclude existence of additional features.

Whilst the principles of the present invention have been set out above in connection with specific embodiments, it is to be understood that this description is merely made by way of example and not as a limitation of the scope of protection which is determined by the appended claims.

1. A computer implemented method for providing a case mix schedule for a plurality of patients in at least one operating room, wherein the method comprises the steps of:
providing an operating room model, wherein the operating room model comprises:
historical patient data comprising at least one patient parameter related to the patient;
medical procedure data comprising at least a procedure identifier indicative for the type of medical procedure and a procedure resource parameter being indicative of the resource requirement of a medical procedure, such as duration and/or other resource requirements of the medical procedure, wherein said procedure resource parameter is associated with at least one patient parameter; and
historical operating room sensor data containing at least one sensor parameter measured during a medical procedure associated with at least one patient parameter and/or procedure identifier;
providing patient data for the plurality of patients, wherein the patient data at least comprises patient parameters for the respective patients;
providing operating room sensor data comprising at least one sensor parameter; and
providing the case mix schedule for the plurality of patients on the basis of the operating room model, the patient data and the operating room sensor data.

2. The method according to claim 1, wherein the step of providing operating room sensor data comprises measuring operating room sensor data is said operating room.

3. The method according to claim 1, wherein the step of providing the case mix schedule comprises updating, on the basis of an updated operating room model and/or operating room sensor data, the case mix schedule.

4. The method according to claim 2, wherein the step of providing the case mix schedule comprises updating, on the basis of an updated operating room model and/or operating room sensor data, the case mix schedule, and wherein the case mix schedule is updated on the basis of measured operating room sensor data, the medical procedure data, the historical patient data or the historical operating room sensor data.

5. The method according to claim 1, further comprising the step of comparing the sensor parameters of the obtained operating room sensor data and historical operating room sensor data and, in dependence on a measured difference, updating the case mix schedule.

6. The method according to claim 1, wherein the medical procedure data comprises, for a plurality of medical procedures, an historical parameter being indicative of the outcome of a medical procedure, such as success and/or duration of the medical procedure, wherein said historical parameter is associated with at least one patient parameter and/or procedure identifier.

7. The method according to claim 6, wherein at least one sensor parameter measured during a medical procedure is associated with at least one patient parameter and an historical parameter.

8. The method according to claim 1, wherein the medical procedure data comprise related procedure data, wherein at least one related procedure associated with a related procedure identifier is associated with a medical, and wherein the related procedure data comprises for a plurality of related procedures at least one related procedure resource parameter, indicative of the required resources of the related procedure, associated with a patient parameter.

9. The method according to claim 1, wherein the operating room model further comprises process map data indicative of steps, including possible alternative steps, for a type of medical procedure, wherein the process map data comprises, associated with at least a procedure identifier, a plurality of steps, hierarchical data representative of the hierarchical order of said steps and at least one step resource parameter associated with at least one step indicative of a resource requirement, for said step, and wherein the case mix schedule is also based on the process map data.

10. The method according to claim 9, wherein the process map data further comprises probability parameters associated with at least two alternative steps.

11. The method according to claim 10, wherein said probability parameter is associated with a patient parameter.

12. The method according to claim 9, wherein said probability parameter is associated with at least one sensor parameter.

13. The method according to claim 1, wherein the operating room model comprises a predictive model.

14. The method according to claim 1, wherein the step of providing the pre-operative plan comprises providing an optimization parameter of the plurality of parameters in said operating room model, and wherein the step of providing the case mix schedule comprises minimizing said optimization parameter in said operating room model on the basis of the received data.

15. The method according to claim 1, further comprising the steps of updating the operating room model on the basis of the received operating room sensor data comprising the at least one sensor parameter.

16. The method according to claim 1, wherein the step of providing the operating room model comprises further comprises the step of providing an operating log indicative of the plurality medical procedures on the plurality of patients in said operating room and determining, on the basis of said operating log, said procedure resource parameters.

17. The method according to claim 16, wherein the step of providing the operating log comprises at least partly determining said log on the basis of the operating room sensor data.

18. The method according to claim 1, wherein the medical procedure data comprises, for a plurality of medical procedures associated with a procedure identifier, an active time parameter representative of the duration a resource is required for said medical procedure and a turnaround parameter, representative of the required turnaround time between procedures wherein said resource is not required, wherein the step of providing the case mix schedule comprises providing, in said planning, predicted active time parameters and turnaround parameters for the at least one operating room.

19. The method according to claim 18, further comprising the step of providing, on the basis of the active time parameters and the turnaround parameters for a planning, an active time ratio indicative of the ratio of the active time parameter and the turnaround parameter, wherein the step of providing the planning comprises optimizing for the active time ratio.

20. The method according to claim 9, wherein the medical procedure data comprises, for a plurality of medical procedures associated with a procedure identifier, an active time parameter representative of the duration a resource is required for said medical procedure and a turnaround parameter, representative of the required turnaround time between procedures wherein said resource is not required, wherein the step of providing the case mix schedule comprises providing, in said planning, predicted active time parameters and turnaround parameters for the at least one operating room; and wherein the active time parameters and turnaround parameter are associated with at least two steps of the process data.

21. A method for providing an operating room model, in particular for use in the method according to claim 1, wherein the method comprises the steps of:
providing historical patient data comprising at least one patient parameter related to the patient;
providing medical procedure data comprising at least a procedure identifier indicative for the type of medical procedure and a procedure resource parameter being indicative of the resource requirement of a medical procedure, wherein said procedure resource parameter is associated with at least one patient parameter;
providing historical operating room sensor data containing at least one sensor parameter measured during a medical procedure associated with at least one patient parameter and/or procedure identifier, and
including the historical patient data, the medical procedure data and the historical operating room sensor data in an operating room model.

22. A computer program product comprising a computer-executable program of instructions for performing, when executed on a computer, the steps of the method of claim 1.

23. A system for providing a case mix schedule for a plurality of patients in at least one operating room, according to the method of claim 1, wherein the system comprises:
a model storage unit arranged for storing an operating room model, wherein the operating room model comprises:
historical patient data comprises at least one patient parameter related to the patient;
medical procedure data comprising at least a procedure identifier indicative for the type of medical procedure and a procedure resource parameter being indicative of the resource requirement of a medical procedure, wherein said procedure resource parameter is associated with at least one patient parameter;
historical operating room sensor data containing at least one sensor parameter measured during a medical procedure associated with at least one patient parameter and/or procedure identifier;
a patient data input unit arranged to receive patient data for the plurality of patients, wherein the patient data at least comprises patient parameters for the respective patients;
a sensor data input unit arranged to receive operating room sensor data comprising at least one sensor parameter;
a processing unit arranged to provide the case mix schedule for the plurality of patients on the basis of the operating room model, the patient data and the operating room sensor data; and
an output unit for outputting the case mix schedule.
